(19) United States
(12) Patent Application Publication
(10) Pub. No.: US 2020/0146972 A1
(43) Pub. Date: May 14, 2020

(54) METHODS FOR ENHANCING EYEBROW APPEARANCE

(71) Applicant: RMV TRADEMARKS, LLC, Auburn Hills, MI (US)

(72) Inventors: Renata Marie Vestevich, Auburn Hills, MI (US); Arun Nandagiri, Libertyville, IL (US)

(21) Appl. No.: 16/191,148

(22) Filed: Nov. 14, 2018

(51) Int. Cl. A61K 8/9789; A61K 8/49; A61K 8/34; A61Q 5/12

(52) U.S. Cl. CPC A61K 8/9789; A61K 8/4973; A61Q 5/12; A61K 8/34; A61K 8/345

(57) ABSTRACT

Compositions comprising from about 0.5 to about 40 percent by weight of an extract of black cohosh, from about 0.5 to about 20 percent by weight of nonionic surfactant, and from about to 40 about 99 percent by weight of hydroxylated solvent are effective in enhancing eyebrow appearance.

### BACKGROUND

Eyebrows play a prominent role in verbal and non-verbal communication, including facial expression of emotions and romantic interest. The physical appearance of eyebrows also elicits strong perceptions regarding youth, beauty, and vitality. Accordingly, eyebrows have long been an important target of cosmetic treatments. Evidence suggests that Cleopatra darkened her eyebrows with carbon. During the 1700s in Western Europe, full eyebrows were considered so essential to facial beauty that some women affixed mouse hide to their foreheads. And the perceived importance of the eyebrows for enhancing beauty continues to the present day.

Practitioners such as make-up artists and cosmetic surgeons have long appreciated the influence of eyebrows on perceptions of attractiveness. Currently, it is common practice to apply colorant-based cosmetics to alter eyebrow color and darkness. Moreover, several cosmetic surgery procedures, including botulinum toxin (“Botox”) injection, microblading, permanent tattooing, and surgical alterations (such as eyebrow transplants) specifically target the appearance of the eyebrows. Problems with all of these techniques persist, however.

Cosmetics containing colorants or other ingredients intended to alter eyebrow appearance are affected by perspiration and precipitation, and in any event need to be frequently re-applied Eyebrow tattooing that adds permanent pigment to the skin is time-consuming, expensive, and often painful because it is performed by a machine with relatively thick needles. The pigment can also migrate beyond the intended area, giving less-than-desirable results. Microblading involves addition of pigment to skin via a handheld tool to produce the appearance of hair strokes that are intended to blend in with existing eyebrow hair. However, microblading deposits pigment into the upper region of the dermis only, so it tends to fade relatively rapidly due to natural regeneration of the dermis.

As the discussion above demonstrates, previously proposed treatments for enhancing eyebrow appearance are unsatisfactory in many respects. The present invention has been shown to improve eyebrow appearance in a non-invasive manner and produce sustainable, positive changes in density, fullness, shine, strength, and overall eyebrow appearance.

### SUMMARY

In one aspect, the present invention provides a composition comprising from about 0.5 to about 40 percent by weight of black cohosh extract, from about 0.5 to about 20 percent by weight of nonionic surfactant, and from about to 40 about 99 percent by weight of hydroxylated solvent.

In another aspect, the invention provides a method for enhancing eyebrow appearance comprising use of a composition comprising from about 0.5 to about 40 percent by weight of an extract of black cohosh, from about 0.5 to about 20 percent by weight of nonionic surfactant, and from about to 40 about 99 percent by weight of hydroxylated solvent.

Embodiments of the present invention may pertain to one or more of these aspects, or to other aspects disclosed herein. These and other aspects and advantages of the present invention will be apparent to those of ordinary skill in the art from the disclosure herein, and in particular from the following detailed description of the invention.

### DETAILED DESCRIPTION

It has been surprisingly found that certain compositions containing an extract of black cohosh when applied to healthy eyebrow hair, i.e., eyebrow hair that has not been compromised by, for example, chemical insults such as chemotherapy, are effective in improving to a statistically significant degree such characteristics as density, fullness, shine, strength, and overall eyebrow appearance.

Compositions of the present invention comprise from about 0.5 to about 40 percent by weight of an extract of black cohosh, from about 0.5 to about 20 percent by weight of nonionic surfactant, and from about to 40 about 99 percent by weight of hydroxylated solvent. The compositions disclosed herein may also optionally include one or more additives. Compositions of the invention may be in any form suitable for topical administration, including, but not limited to, liquids, gels, pastes, or emulsions.

As used herein, the term “an extract of black cohosh” refers to an alcoholic or glycolic extract from the roots and/or rhizomes of Actaea racemosa L. (black cohosh) (syn. Cimicifuga racemosa L.) As used herein, the term “extract” refers to a concentrate of water-soluble and/or alcohol-soluble plant components from the portion of the plant extracted. As used herein, the term “alcoholic extract” refers to the product of extraction using one or more compounds having one or more hydroxyl groups, such as isopropyl alcohol. As used herein, the term “glycolic extract” refers to the product of extraction using one or more compounds having two hydroxyl groups, such as propylene glycol.

Black cohosh has a lengthy and varied history of medicinal use. It was traditionally used by Native Americans and early colonists to treat various conditions including malaria, rheumatism, renal abnormalities, tonsillitis, menstrual cramping, and menopausal symptoms. In recent decades, the roots and rhizomes of this plant have been used in dietary supplements and homeopathic remedies.

Black cohosh is a member of the botanical family Ranunculaceae (buttercup family), and is a perennial plant native to temperate regions of the Northern Hemisphere. The taxonomic genus of the plant species black cohosh is a matter of dispute. In the eighteenth century, the Swedish botanist Carl Linnaeus classified the species within the genus Actaea. This designation was revised in the nineteenth century by the English botanist Thomas Nuttall, who reclassified the species into the genus Cimicifuga. Recent data from morphological and gene phylogeny analyses suggest that black cohosh is more closely related to species of the genus Actaea than to other Cimicifuga species, prompting some botanists to revise the black cohosh genus classification to Actaea racemosa as originally proposed by Linnaeus. Scientific debate regarding the proper taxonomic designation for black cohosh continues to this day. Accordingly, black cohosh is known as both Actaea racemosa and Cimicifuga racemosa. Other common names include baneberry, black snakeroot, black bugbane, bugbane, bugwort, fairy candle, macrotys, rattleroot, rattletop, rattleweed, and squawroot. Insects avoid the black cohosh plant, which accounts for some of its common names.

The genus Cimicifuga includes eighteen species, one of which is native to Europe, six of which are native to North America, and the remainder of which are native to northeast Asia. The genera Actaea and Cimicifuga when combined include 25 to 30 species. Thus, the black cohosh genus contains numerous species, and any suitable black cohosh plant within the black cohosh genus can be used as the source of the extract of black cohosh used in compositions of the present invention.

The mechanism of action of extracts of black cohosh has not been identified with certainty, and the biological activity of extracts of black cohosh cannot as yet be attributed to any one chemical component or group of components. Nevertheless, the activity of extracts of black cohosh is generally believed to derive from the compounds cimifugin and/or cimiracemoside C.

The major secondary compounds in extracts of black cohosh include triterpene glycosides and phenolics. Typically present triterpene glycosides include cimiracemoside F, 23-epi-26-deoxyactein (previously known as 27-deoxyactein), and actein. Because triterpene glycosides are the major secondary compounds of black cohosh, many commercial black cohosh products are standardized to triterpene glycosides, most often to 23-epi-26-deoxyactein. In particular, extracts of black cohosh are commonly standardized to contain 1 mg of total triterpenes (expressed as 23-epi-26-deoxyactein) in each 20-mg dose of extract, i.e., at least about 5 weight percent of triterpenes. Typical phenolic constituents of extracts of black cohosh include caffeic acid, fukiic acid, piscidic acid, and their derivatives

Black cohosh extract used in compositions of the present invention can be obtained from one or more portions of the black cohosh plant. In the present invention, the black cohosh extract is preferably taken from the root and rhizome of the plant. The black cohosh plant can be prepared for extraction by subdividing the plant into small pieces. The small pieces are ground into a powder form, such as by mechanical disruption in a blender or other similar means. If less than the whole plant is being used, the desired portions are first isolated from the remaining portions of the plant and then subdivided into small pieces and ground into a powder form.

Extracts of black cohosh extract can be prepared using any one or more extraction techniques which are well known to an ordinarily skilled artisan. Suitable supercritical extraction methods are disclosed in U.S. Pat. Nos. 5,932,101 and 5,120,558, both of which are incorporated by reference herein. Suitable conventional extraction techniques are disclosed in U.S. Pat. Nos. 5,891,440, 5,874,084, and 5,908,628, all of which are incorporated by reference herein.

A preferred but non-limiting extraction technique involves an alcoholic extraction, whereby a lower alkyl alcohol such as isopropanol is contacted with the roots and/or rhizomes of black cohosh to produce a liquid extract termed an alcoholic extract. Optionally, water can be additionally present during extraction to produce a liquid extract termed an aqueous alcoholic extract.

Another preferred but non-limiting extraction technique involves a glycolic extraction, whereby a diol such as propylene glycol is contacted with the roots and/or rhizomes of black cohosh to produce a liquid extract termed a glycolic extract. Optionally, water can be additionally present during extraction to produce a liquid extract termed an aqueous glycolic extract.

Other preferred but non-limiting extraction techniques involve the use of a combination of at least one alcohol and at least one glycol, with or without water being additionally present.

The resulting extract of black cohosh obtained by any of these or other well-known techniques may be maintained as a liquid or converted to another form using any convenient means well known to an ordinarily skilled artisan, such as a dried powder for later reconstitution. In some instances, the extract of black cohosh extract may be supplied as an aqueous solution. In other instances, the extract black cohosh extract may be supplied as a solution of water and an alcohol such as isopropanol. In still other instances, the extract of black cohosh extract may be supplied as a solution of water and a hydroxylated solvent such as propylene glycol. In still other instances, the extract of black cohosh extract may be supplied as a solution of water, an alcohol such as isopranol, and a hydroxylated solvent such as propylene glycol.

A particularly preferred but non-limiting exemplary extract of black cohosh is a material known by the brand name Actiphyte of Black Cohosh, available commercially as a light to medium amber liquid comprising 39.5 weight percent of propylene glycol, 39.5 weight percent of water, 20 weight percent of Cimicifuga Racemosa root extract, and 1.0 weight percent of a preservative.

Compositions suitable for use in the methods described herein may contain from about 0.5 to about 40 percent by weight of extract of black cohosh, and more particularly from about 2 to about 35 percent by weight of extract of black cohosh. More preferable compositions contain from about 4 to about 25 percent by weight of extract of black cohosh.

### Nonionic Surfactant

As used herein, the term “nonionic surfactant” refers to any surfactant lacking a charge in its hydrophilic portion. Also as used herein, the term “nonionic surfactant” refers to any suitable nonionic surfactant or mixture of two or more nonionic surfactants.

Nonionic surfactants are well known in the art, and the present invention may employ any nonionic surfactant or mixture thereof that is suitable for application to skin or hair and is compatible with other ingredients present in the composition. Suitable nonionic surfactants are described in McCutcheon's, Detergents and Emulsifiers, North American edition (1986), published by allured Publishing Corporation; McCutcheon's, Functional Materials, North American Edition (1992); International Cosmetic Ingredient Dictionary and Handbook, 7^{th} ed. (1997), published by the Cosmetic, Toiletry, and Fragrance Association; and U.S. Pat. No. 3,929,678 issued to Laughlin, et al on Dec. 30, 1975, all of which are incorporated by reference herein.

Most commonly, nonionic surfactants are compounds produced by the condensation of an alkylene oxide (hydrophilic in nature) with an organic hydrophobic compound which is usually aliphatic or alkyl aromatic in nature. The length of the hydrophilic or polyoxyalkylene moiety which is condensed with any particular hydrophobic compound can be readily adjusted to yield a water-soluble compound having the desired degree of balance between hydrophilic and hydrophobic elements. Another variety of nonionic surfactant is the semi-polar nonionic typified by the amine oxides, phosphine oxides, and sulfoxides.

Non-limiting examples of nonionic surfactants suitable for use in the present invention include fatty alcohols (including, but not limited to, cetyl alcohol, stearyl alcohol, cetostearyl alcohol, and oleyl alcohol); polyoxyethylene glycol alkyl ethers (including but not limited to polyoxyethylene lauryl ether (Brij® 35)); polyoxypropylene glycol alkyl ethers; glucoside alkyl ethers; polyoxyethylene glycol octylpohenol ethers (including, but not limited to, Triton® X-100); polyoxyethylene glycol alkylphenol ethers (including, but not limited to, nonoxynol-9); glycerol alkyl esters; polyoxyethylene glycol sorbitan alkyl esters (including, but not limited to, polysorbate-20 and polysorbate-80); sorbitan alkyl esters (including, but not limited to, Span®-20 and Span®-80); block copolymers of polyethylene glycol and polypropylene glycol (including, but not limited to, poloxamer-407); amine oxides; and sulfoxides.

Preferred nonionic surfactants for use in compositions of the present invention are polyoxyethylene glycol sorbitan alkyl esters. Most preferably, the nonionic surfactant is a polyoxyethylene derivative of sorbitan monolaurate such as polysorbate 20. The compositions of the invention may contain from about 0.5 to about 20 percent by weight of a nonionic surfactant, and more particularly from about 1 to about 10 percent by weight of a nonionic surfactant. More preferably, compositions of the invention contain from about 4 to about 6 percent by weight of a nonionic surfactant.

### Hydroxylated Solvent

Compositions of the present invention include one or more hydroxylated solvents. As used herein, the term “hydroxylated solvent” refers to any solvent or mixture of solvents containing at least one hydroxyl group. Suitable hydroxylated solvents include water, monohydric alcohols, polyhydric alcohols, or mixtures thereof. Water is a preferred hydroxylated solvent.

Monohydric alcohols that may be used in compositions of the present invention include, but are not limited to, C₁ to C₆ alcohols. Preferred monohydric alcohols include methanol, ethanol, n-propanol, isopropanol, n-butanol, isobutanol, t-butanol, and mixtures thereof. Isopropanol is a preferred monohydric alcohol.

Polyhydric alcohols that may be used in compositions of the present invention include, but are not limited, to glycols such as ethylene glycol, propylene glycol, glycerol, and polyol monoethers. Propylene glycol is a preferred polyhydric alcohol.

Preferably, the hydroxylated solvent comprises a mixture of water and a monohydric alcohol, or a mixture of water and a polyhydric alcohol. Most preferably, the hydroxylated solvent comprises a mixture of water, a monohydric alcohol, and a polyhydric alcohol.

### Optional Components

The compositions of the invention may optionally contain additives, for example preservatives, colorants, agents to adjust pH, and fragrances. Any of the many preservatives well known in the art can be used. Preservatives for use with the invention particularly include, but are not limited, to phenoxyethanol/ethylhexylglycerin, methylparaben, and propylparaben. In certain cases, however, it may be desirable to minimize or eliminate use of paraben-based preservatives to improve consumer acceptance.

The pH of the compositions of the invention may be adjusted to maximize compatibility with skin and/or to increase or decrease the dermal and/or follicular penetration of the composition, as appropriate. In certain embodiments, the pH of the compositions of the invention is from about 4 to about 8. In other embodiments, it may be from about 5 to about 6, or from about 7 to about 8. Any of the many pH-adjusting ingredients well known in the art can be used. The pH-adjusting ingredients for use with the invention particularly include, but are not limited to, citric acid and triethanolamine.

The compositions of the present invention are used to treat eyebrow hair. In particular, the present invention includes methods for enhancing eyebrow appearance by contacting eyebrow hair with compositions as disclosed herein.

All percentages used herein are by weight unless otherwise specified. Various embodiments and/or features are disclosed herein. As will be apparent to the ordinarily skilled practitioner, all combinations of such embodiments and features are possible and many such combinations can result in preferred executions of the invention.

The methods and compositions described herein as representative preferred embodiments are exemplary and are not intended as limitations on the scope of the invention. Modifications thereto will be evident to those skilled in the art, and are encompassed within the spirit of the invention. The terms and expressions which have been employed are used as terms of description and not of limitation, and there is no intention by the use of such terms and expressions to exclude any equivalents of the features shown and described or portions thereof. Thus, it should be understood that although the present invention has been specifically disclosed by embodiments and optional features, modifications and variations of the concepts herein disclosed are considered to be within the scope of this invention as defined by the description and the appended claims.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

In all embodiments, all percentages are by weight of the composition (or formulation), unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25 degrees Centigrade and at ambient conditions, where “ambient conditions” means conditions under about 1 atmosphere of pressure and at about 50% relative humidity.

The use of the terms “a” and “an” and “the” are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. Also, unless the context dictates the contrary, all ranges set forth herein should be interpreted as being inclusive of their endpoints, and open-ended ranges should be interpreted to include only commercially practical values.

Ingredients of compositions describe herein can be combined in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The present invention may be better understood in light of the following examples, which are intended for illustration purposes only, and should not be construed as limiting the scope of the invention in any way.

### EXAMPLES

### 1. Formulation

A composition was prepared by traditional methods known in the art, as follows:

| Ingredient | Function | Wt % |
|---|---|---|
| Water | Diluent/Carrier | 54.7 |
| Isopropanol | Diluent/Carrier | 15.0 |
| Polysorbate 20 | Surfactant | 5.0 |
| Actyphyte of Black Cohosh, | Active Extract | 25.0 |
| supplied as 20% solution of | | |
| Cimicifuga Racemosa root | | |
| extract in a 1:1 mixture | | |
| of propylene glycol and water | | |
| Euxyl PE 9010 (Phenoxyethanol/ | Preservative | 0.3 |
| Ethylhexylglycerin) | | |
| Citric Acid | pH Adjuster | QS pH |
| | | 5.0-6.0 |

### 2. Administration of Formulations to Subjects

Efficacy of composition 1 described above was evaluated in 44 subjects. Each subject topically applied the composition to each eyebrow twice daily (with some infrequent missed applications) for sixteen weeks.

The formulation was stored at room temperature in a tightly closed bottle between usage periods.

At interim periods of four weeks and ten weeks, and at the conclusion of treatment after 16 weeks, the efficacy of the treatment was evaluated by each subject on the parameters of density, fullness, shine, strength, and overall eyebrow appearance. The data is summarized as follows:

| Improvement | Eyebrow | Eyebrow | Eyebrow | Eyebrow | Overall |
|---|---|---|---|---|---|
| Attributes | Density | Fullness | Shine/Suppleness | Strength | Appearance |
| Any Improvement | 36/44 | 34/44 | 31/44 | 34/44 | 37/44 |
| % Out of N = 44 | 81.8% | 77.2% | 70.4% | 77.2% | 84% |

IMPROVEMENT ATTRIBUTES

| | 4 WEEKS | 10 WEEKS | p-Value | Stat Code |
|---|---|---|---|---|
| Eyebrow Density | 1.77 | 2.09 | 3.85E−02 | S |
| Eyebrow Fullness | 1.86 | 2.27 | 2.39E−02 | S |
| Eyebrow Shine | 1.82 | 2.23 | 3.93E−02 | S |
| Eyebrow Strength | 1.80 | 2.23 | 3.05E−02 | S |
| Overall Eyebrow | 2.09 | 2.57 | 1.53E−02 | S |
| Appearance | | | | |

IMPROVEMENT ATTRIBUTES

| | 4 WEEKS | 16 WEEKS | p-Value | Stat Code |
|---|---|---|---|---|
| Eyebrow Density | 1.77 | 2.39 | 7.40E−04 | S |
| Eyebrow Fullness | 1.86 | 2.43 | 5.11E−03 | S |
| Eyebrow Shine | 1.82 | 2.30 | 2.24E−02 | S |
| Eyebrow Strength | 1.80 | 2.57 | 4.22E−04 | S |
| Overall Eyebrow | 2.09 | 2.61 | 9.79E−03 | S |
| Appearance | | | | |

IMPROVEMENT ATTRIBUTES

| | 10 WEEKS | 16 WEEKS | p-Value | Stat Code |
|---|---|---|---|---|
| Eyebrow Density | 2.09 | 2.39 | 0.12 | P |
| Eyebrow Fullness | 2.27 | 2.43 | 0.45 | P |
| Eyebrow Shine | 2.23 | 2.30 | 0.75 | P |
| Eyebrow Strength | 2.23 | 2.57 | 0.13 | P |
| Overall Eyebrow | 2.57 | 2.61 | 0.83 | P |
| Appearance | | | | |

S = statistically significant at the 95% confidence level
P = statistical parity at the 95% confidence level

As the above examples demonstrate, at the end of the study, greater than 70% of the study participants reported noticeable improvement on all of the attributes of density, fullness, shine, strength, and overall eyebrow appearance.

As the above examples demonstrate, at the interim period of ten weeks after the start of the study, the subjects reported a statistically significant improvement as compared to the interim period of four weeks on all of the attributes of density, fullness, shine, strength, and overall eyebrow appearance.

As the above examples demonstrate, at the conclusion of the study after sixteen weeks, the subjects reported a statistically significant improvement as compared to the interim period of four weeks on all of the attributes of density, fullness, shine, strength, and overall eyebrow appearance.

As the above examples demonstrate, comparing the evaluations after the interim period of ten weeks and at the conclusion of the study after sixteen weeks, the improvements on all of the attributes of density, fullness, shine, strength, and overall eyebrow appearance were at statistical parity, indicating that the statistically significant improvements on these attributes that were observed after ten weeks were maintained at sixteen weeks.

1. A method for enhancing eyebrow appearance, comprising contacting a subject's eyebrow hair with a composition comprising:
(a) from about 0.5 to about 40 percent by weight of an extract of black cohosh;
(b) from about 0.5 to about 20 percent by weight of nonionic surfactant; and
(c) from about 40 to about 99 percent by weight of hydroxylated solvent.

2. The method of claim 1, wherein the extract of black cohosh is selected from the group consisting of an aqueous alcoholic extract of black cohosh or an aqueous glycolic extract of black cohosh.

3. The method of claim 2, wherein the extract of black cohosh comprises at least about 10 weight percent of Cimicifuga Racemosa root extract.

4. The method of claim 3, wherein the extract of black cohosh comprises an alcohol and water in a ratio of from about 1:5 to about 5:1.

5. The method of claim 1, wherein the extract of black cohosh comprises the compound cimiracemoside C, at least one triterpene compound, and at least one phenolic compound.

6. The method of claim 5, wherein the extract of black cohosh comprises at least about 5 weight percent of total triterpene compounds.

7. The method of claim 5, wherein the extract of black cohosh comprises 23-epi-26-deoxyactein.

8. The method of claim 1, wherein the composition has a pH of from about 4 to about 8.

9. The method of claim 1, wherein the nonionic surfactant comprises a polyoxyethylene derivative of sorbitan monolaurate.

10. The method of claim 9, wherein the polyoxyethylene derivative of sorbitan monolaurate is polysorbate 20.

11. The method of claim 1, wherein the nonionic surfactant comprises a mixture of at least two nonionic surfactants.

12. The method of claim 11, wherein the nonionic surfactant comprises a mixture of polysorbate 20 and at least one other nonionic surfactant.

13. The method of claim 1, wherein the hydroxylated solvent is water.

14. The method of claim 1, wherein the hydroxylated solvent comprises a monohydric alcohol.

15. The method of claim 14, wherein the monohydric alcohol comprises a C1 to C6 alcohol.

16. The method of claim 15, wherein the monohydric alcohol is isopropanol.

17. The method of claim 15, wherein the hydroxylated solvent comprises a polyhydric alcohol.

18. The method of claim 17, wherein the polyhydric alcohol is selected from the group consisting of ethylene glycol, propylene glycol, glycerol, polyol monoethers, and mixtures of two or more thereof.

19. The method of claim 17, wherein the polyhydric alcohol is propylene glycol.

20. The method of claim 1, wherein the hydroxylated solvent comprises a mixture of water and a monohydric alcohol.

21. The method of claim 1, wherein the hydroxylated solvent comprises a mixture of water and a polyhydric alcohol.

22. The method of claim 1, wherein the hydroxylated solvent comprises a mixture of water, a monohydric alcohol, and a polyhydric alcohol.
